(19) United States
(12) Patent Application Publication
(10) Pub. No.: US 2025/0009741 A1
(43) Pub. Date: Jan. 9, 2025

(54) NOVEL COMPOSITIONS AND METHODS

(71) Applicant: INTRA-CELLULAR THERAPIES, INC., New York, NY (US)

(72) Inventors: Peng LI, New Milford, NJ (US); Robert DAVIS, San Diego, CA (US)

(21) Appl. No.: 18/893,190

(22) Filed: Sep. 23, 2024

Related U.S. Application Data
(63) Continuation of application No. 17/271,965, filed on Feb. 26, 2021, now Pat. No. 12,144,808.
(60) Provisional application No. 62/724,472, filed on Aug. 29, 2018.

(51) Int. Cl. A61K 31/4985; A61K 9/70

(52) U.S. Cl. CPC A61K 31/4985; A61K 9/7084

(57) ABSTRACT

The disclosure provides new transdermal pharmaceutical compositions comprising 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one or 1-(4-fluoro-phenyl)-4-((6bR,10aS)-2,2-d₂-3-methyl-2,3,6b,9,10,10a-hexahydro-1H,7H-pyrido[3′,4′:4,5]pyrrolo[1,2,3-de]quinoxalin-8-yl)-butan-1-one or comprising—(4-fluoro-phenyl)-4-((6bR,10aS)-1,1,2,2-d₄-3-methyl-2,3,6b,9,10,10a-hexahydro-1H,7H-pyrido[3′,4′:4,5]pyrrolo[1,2,3-de]quinoxalin-8-yl)-butan-1-one, in free base, co-crystal or salt form, together with methods of making and using them.

### CROSS REFERENCE TO RELATED APPLICATIONS

This international application claims priority to and the benefit of U.S. Provisional Application No. 62/724,472, filed Aug. 29, 2018, the contents of which is incorporated herein by reference in its entirety.

### FIELD

This disclosure relates to certain novel transdermal pharmaceutical formulations comprising substituted heterocycle fused gamma-carbolines, the manufacture of such formulations, and methods of use thereof, e.g., in the treatment of diseases or abnormal conditions involving or mediated by the 5-HT_{2A} receptor, serotonin transporter (SERT), and/or dopamine D₁/D₂ receptor signaling pathways. The invention includes methods of treatment and/or prophylaxis of diseases and disorders including, but not limited to, anxiety, psychosis, schizophrenia, sleep disorders, sexual disorders, migraine, conditions associated with cephalic pain, social phobias, gastrointestinal disorders such as dysfunction of the gastrointestinal tract motility and obesity; depression (including major depressive disorder (MDD)) and mood disorders associated with psychosis or Parkinson's disease; psychosis such as schizophrenia associated with depression; bipolar disorder (e.g., bipolar depression); and other psychiatric and neurological conditions, as well as to combinations with other agents.

### BACKGROUND

1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (sometimes referred to as 4-((6bR,10aS)-3-methyl-2,3,6b,9,10,10a-hexahydro-1H-pyrido[3′,4′:4,5]pyrrolo[1,2,3-de]quinoxalin-8(7H)-yl)-1-(4-fluorophenyl)-1-butanone, or as ITI-007 or lumateperone), has the following structure:

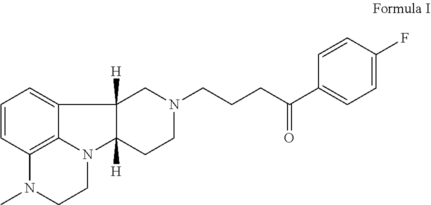 [SMILES: CN1CCN2c3c(cccc31)[C@@H]1CN(CCCC(=O)c3ccc(F)cc3)CC[C@@H]12]

The Compound of Formula I is a potent 5-HT_{2A} receptor ligand (Ki=0.5 nM) with strong affinity for dopamine (DA) D2 receptors (Kᵢ=32 nM) and the serotonin transporter (SERT) (Kᵢ=26 nM, measured using 3H-imipramine binding displacement to human recombinant SERT), but negligible binding to receptors associated with cognitive and metabolic side effects of antipsychotic drugs (e.g., H1 histaminergic, 5-HT_{2C}, and muscarinic receptors). This compound is currently in clinical trials, i.e., for the treatment of schizophrenia, bipolar disorder and dementia including Alzheimer's disease. The Compound of Formula I, and analogs thereof, salts thereof, and methods of treatment comprising such compounds, and methods of manufacturing such compounds, have been disclosed, e.g., in U.S. Pat. Nos. 6,548,493; 7,238,690; 6,552,017; 6,713,471; 7,183,282; RE39,680; RE39,679; U.S. Patent Publications 2004/209864, 2010/113781, 2011/071080, 2011/112105, 2013/0202692, 2015/0079172, 2017/0183350; and PCT Publication WO 2017/165843 (and US 2019/0231780) and WO 2017/117514 (Tung, R. D.). The contents of each of these U.S. Patents, U.S. Patent Publications, and PCT Publications are hereby incorporated by reference in their entireties.

Deuterated variants of ITI-007 are generally disclosed in US 2017/0183350 and WO 2017/165843 (US 2019/0231780). The deuterated compounds are designed to slow or inhibit in vivo metabolism by substituting deuterium atoms for hydrogen atoms of ITI-007 at molecular positions which are the target of metabolic activity. The natural metabolites of ITI-007 are pharmacologically active, but with somewhat different receptor selectivity profiles. These deuterated derivatives can therefore provide modified pharmacokinetic profiles owing to altered rates or pathways of metabolism, as well as modified overall pharmacological profile due to shifting the balance between active parent species and active metabolite species.

One such deuterated compound is 1-(4-fluoro-phenyl)-4-((6bR,10aS)-2,2-d₂-3-methyl-2,3,6b,9,10,10a-hexahydro-1H,7H-pyrido[3′,4′:4,5]pyrrolo[1,2,3-de]quinoxalin-8-yl)-butan-1-one, the Compound of Formula II:

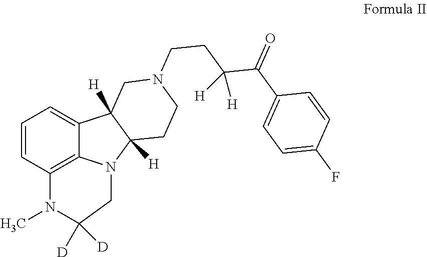 [SMILES: [2H]C1([2H])CN2c3c(cccc3N1C)[C@@H]1CN(CCCC(=O)c3ccc(F)cc3)CC[C@@H]12]

Another such deuterated compound is 1-(4-fluoro-phenyl)-4-((6bR,10aS)-1,1,2,2-d₄-3-methyl-2,3,6b,9,10,10a-hexahydro-1H,7H-pyrido[3′,4′:4,5]pyrrolo[1,2,3-de]quinoxalin-8-yl)-butan-1-one, the Compound of Formula III:

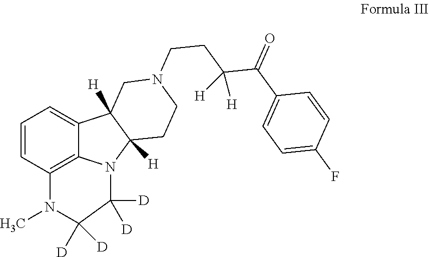 [SMILES: [2H]C1([2H])N(C)c2cccc3c2N([C@H]2CCN(CCCC(=O)c4ccc(F)cc4)C[C@@H]32)C1([2H])[2H]]

The Compounds of Formula I, II and Formula III each undergo significant first-pass metabolism in the liver as well as significant intraluminal metabolism. For example, it has been found that the oral bioavailability of the compound of Formula I is about 5% or less. The high rate of metabolism requires the administration of higher oral doses of drug than would otherwise be needed, resulting in an increased burden on the liver, increased costs in manufacturing, difficulties in formulation and potentially higher patient-to-patient variability in dose response. There is, therefore, a need for new routes of administration that avoid first-pass hepatic metabolism and GI luminal metabolism, and which would result in correspondingly lower dosing requirements.

It has been disclosed that for a number of drugs, transdermal delivery, such as transdermal patches, transdermal gels or ointments, and transdermal sprays, are effective alternatives to traditional dosage forms such as parenteral and oral dosing. Parenteral (intravenous) dosing is very effective in avoiding first-pass and GI metabolism but is limited in its usefulness because it requires administration by trained professionals, usually in a clinical environment. In contrast, transdermal delivery systems can be used to formulate drugs which can be taken by patients without professional supervision and can result in steady, long-term drug absorption with minimal first-pass metabolism.

The use of transdermal drug delivery formulations is well known, with transdermal patch formulations of scopolamine dating back to 1979. These formulations involve the transfer of active drug agent across and through the epidermal and dermal layers of the skin to reach the capillaries deep within the dermis, which is distinct from dermal drug delivery, in which a drug active is merely delivered to the skin itself. Transdermal drug delivery is a method of systemic drug administration, not local administration. Transdermal drug delivery is difficult to achieve due to the natural impermeability of the skin to foreign substances, including water. Transdermal drug delivery is thus particularly difficult for water-soluble drug substances. In contrast to transdermal drug delivery is transmucosal drug delivery, in which a drug substance is transported across a mucous membrane, including the oral mucosa, nasal mucosa, and the vaginal mucosa. Like transmucosal drug delivery, trans-dermal drug delivery has the distinct advantage of avoiding first-pass hepatic and GI metabolism, and it is also patient friendly as the common formulations, such as patches, can be applied by patients and replaced on an infrequent basis. Many trans-dermal drug delivery systems can be optimized to ensure long-term (e.g., days or weeks) of steady, stable systemic drug delivery.

Existing transdermal delivery systems include patches, gels, ointments and sprays. Examples of such delivery systems include those disclosed in U.S. Pat. No. 9,993,466 to Lee et al., U.S. Pat. No. 9,913,840 to Jain et al., U.S. Pat. No. 9,693,970 to Mo, U.S. Pat. No. 9,585,862 to Hwang et al., and U.S. Pat. No. 7,858,114 to Ito. Individual formulations must be fine-tuned to particular active pharmaceutical ingredients to ensure accuracy and reliability in delivery. Thus, while the field of transdermal drug delivery has a long history, considerably effort is required in adapting any selected transdermal delivery technology to a particular active pharmaceutical ingredient.

The use of transdermal drug delivery is particularly useful when treating patients who are or are expected to be unreliable in daily dosing with oral medications. Foremost among such patients are those with psychiatric or neurological illnesses, such as psychosis, schizophrenia and cognitive and memory disorders (e.g., dementias, such as Alzheimer's disease).

Compounds of Formula I, II and III in free base form are hydrophobic and have good lipid-solubility, but they are difficult to crystallize and tend to exist as oils. In contrast, Compounds of Formula I, II and III may exist in stable, solid crystal salt forms which are easy to handle, but are not lipid-soluble. As a result, developing a transdermal formulation of these compounds may not be an easy undertaking.

There is a need for improved pharmaceutical delivery systems for the safe, effective, reliable delivery of the Compounds for Formula I and/or the Compound of Formula II, especially for long-term treatment of patients for whom daily oral dosing is unreliable. The present disclosure provides novel transdermal formulations for the delivery of these compounds without the drawbacks of existing parenteral and oral delivery systems.

### BRIEF SUMMARY

The present disclosure is directed to novel transdermal pharmaceutical formulations comprising 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (lumateperone), as well as deuterated variants thereof, in free base, co-crystal or salt forms. Transdermal formulations include, but are not limited to patches, gels, ointments, and sprays.

Further areas of applicability of the present invention will become apparent from the detailed description provided hereinafter. It should be understood that the detailed description and specific examples, while indicating preferred embodiments of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material in free base equivalent form.

In a first embodiment, the present disclosure provides a transdermal pharmaceutical formulation (Formulation 1) comprising 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (Compound of Formula I), in free base, co-crystal or salt form. The present disclosure further provides the following Formulations:
1.1. Formulation 1, wherein the formulation comprises the Compound of Formula I in free base form.
1.2. Formulation 1, wherein the formulation comprises the Compound of Formula I in salt form, e.g., in pharmaceutically acceptable salt form, optionally in amorphous solid or crystal salt form; or in co-crystal form, e.g., in nicotinamide or isonicotinamide co-crystal form.
1.3. Formulation 1.2, wherein the salt form is a tosylate, oxalate, cyclamate, 4-aminosalicylate, or hydrochloride salt form, optionally, wherein said salt form is a crystal salt form.
1.4. Any preceding formulation, wherein the formulation comprises from 1 to 1000 mg of the Compound of Formula I (free base equivalent), e.g., 1 to 750 mg, 1 to 500 mg, 1 to 300 mg, 1 to 200 mg, 10 to 200 mg, 50 to 200 mg, 100 to 200 mg, 100 to 300 mg, 200 to 300 mg, 200 to 500 mg, or 500 to 1000 mg.
1.5. Any preceding formulation, wherein the formulation comprises from 1 to 100 mg of the Compound of Formula I (free base equivalent), e.g., 10 to 70 mg, or 40 to 70 mg.
1.6. Any preceding formulation, wherein the formulation comprises from 1 to 80 mg of the Compound of Formula I (free base equivalent), e.g., from 1 to 50 mg, or from to 5 to 50 mg.
1.7. Any preceding formulation, wherein the composition further comprises one or more excipients, e.g., materials which stabilize the Compound of Formula I, and/or enhance absorption into the skin of the Compound of Formula I.
1.8. Formula 1.7, wherein the one or more excipients are selected from the group consisting of solvents, solubilizers, plasticizers, surfactants, binders, humectants (e.g., polyols), antioxidants, buffering agents (e.g., acids, bases and/or salts thereof), emollients, and thickening agents (e.g., gelling agents).
1.9. Formula 1.7, wherein the one or more excipients are selected from any of the following: alcohols (ethanol, isopropanol, propanol, glycerol, propylene glycol, butylene glycol, pentylene glycol, hexylene glycol, polyethylene glycol), non-alcoholic solvents (e.g., dimethylsulfoxide, dimethylformamide, acetonitrile), gums (e.g., acacia, guar, agar, xanthan, tragacanth, karaya, gellan), polysaccharides and polysaccharide derivatives (e.g., starches, dextrans, pectins, alginates, carrageenans, cellulose, cellulose derivatives (e.g., carboxymethyl cellulose, methylcellulose, hydroxyalkyl celluloses (e.g., hydroxyethyl cellulose, hydroxypropyl cellulose, hydroxypropyl methyl cellulose)), gelatins including non-gelling and gelling types (e.g., mammalian gelatins such as bovine gelatin, porcine gelatins, avian gelatins, fish gelatins (e.g., mixed high molecular weight and low molecular weight gelatins), synthetic polymers (e.g., polyvinylpyrrolidones, polyethylene oxide and/or polypropylene oxide polymers and copolymers (e.g., poloxamers, such as poloxamer 188), polyacrylate polymers (e.g., carbopols), polyamide polymers, sugars and sugar alcohols (e.g., dextrose, lactose, galactose, glucose, ribose, sucrose, trehalose, mannitol, maltitol, lactitol, sorbitol, xylitol, erythritol, galactitol, inositol) polypeptides/proteins, amino acids, inorganic or organic acids (e.g., citric acid, lactic acid, malic acid, mandelic acid, tropic acid, glycolic acid, gluconic acid, benzoic acid, toluenesulfonic acid, phosphoric acid, sulfuric acid, hydrochloric acid, tartaric acid, oxalic acid, cyclamic acid, ascorbic acid, methanesulfonic acid, benzenesulfonic acid, formic acid) and their salts (e.g., sodium, potassium, calcium, magnesium, lithium, ammonium salts of aforementioned acids) and esters (e.g., methyl or ethyl esters, or fatty acid esters), inorganic or organic bases (e.g., alkali metal or alkaline earth metal carbonates, bicarbonates, hydroxide, oxides), anionic surfactants (e.g., sodium lauryl sulfate, sodium laureth sulfate, sodium dodecylbenzenesulfonate, sodium lauroyl sarcosinate, sodium stearate), cationic surfactants (e.g., benzalkonium halides, cetylpyridinium halides, cetrimonium halides, benzethonium halides), zwitterionic surfactants (e.g., cocamidoalkyl betaines, such as cocamidopropyl betaine, or amine oxides, such as dimethyldodecylamine oxide and myristamine oxide), nonionic surfactants (e.g., fatty alcohol ethoxylates (e.g., polyethylene glycol polydodecyl ethers)), sorbitan esters (e.g., sorbitan monolaurate, sorbitan monooleate, sorbitan monopalmitate, sorbitan monostearate, sorbitan tristearate), polyethoxylated sorbitan esters (e.g., polysorbate 20, polysorbate 40, polysorbate 60, polysorbate 80), fatty acid esters (e.g., lauryl lactate, isopropyl myristate, oleyl oleate, methyl laurate, isopropyl palmitate, ethyl oleate), fatty alcohols (e.g., dodecanol, octyldecanol, lauryl alcohol), and antioxidants (e.g., ascorbic acid, citric acid, ascorbyl palmitate, sodium metabisulfite, BHT, BHA, TBHQ, propyl gallate, beta-carotene, tocopherols, tocotrienols).
1.10. Any of Formulations 1.7 to 1.9, wherein any one or more of said excipients are present in an amount of 0.1 to 80% by weight of the formulation, e.g., 0.1 to 60%, or 0.1 to 40%, or 0.1 to 20%, or 0.1 to 15% or 0.1 to 10%.
1.11. Any preceding formulation, wherein the formulation comprises an adhesive polymer, optionally in an amount from 25-80% by weight.
1.12. Formulation 1.11, wherein the adhesive polymer comprises a polymer selected from the group consisting of acrylate polymers or co-polymers (e.g., polymers in which at least one monomeric unit is an acrylic acid or ester, and in which one or more other monomeric units may be a non-acrylate monomer, e.g., a vinyl monomer), polyvinylpyrrolidones (e.g., linear or cross-linked), copolymers of maleic acid or a maleic ester with a vinyl ether (e.g., co-polymer of maleic acid or anhydride with methyl vinyl ether), cellulose derivatives (e.g., carboxymethyl cellulose), silicone polymers (e.g., dimethylsilicone), and mixtures thereof, such as, a mixture of a polyacrylate polymer and a silicone polymer.
1.13. Any preceding formulation, wherein the formulation is an ointment.
1.14. Any of formulations 1 or 1.1 to 1.11, wherein the formulation is a spray.
1.15. Any of formulations 1 or 1.1 to 1.11, wherein the formulation is a gel.
1.16. Any of formulations 1 or 1.1 to 1.11, wherein the formulation is comprised in a patch, e.g., an adhesive patch.
1.17. Formulation 1.16, wherein the patch comprises at least one drug reservoir layer and wherein the formulation is the drug reservoir layer.
1.18. Formulation 1.17, wherein the drug reservoir layer is adhesive.
1.19. Any preceding formulation wherein the Compound of Formula I is incorporated into the formulation as microparticles (e.g., particles having an average diameter of less than 50 μm, less than 30 μm, less than 10 μm, or less than 5 μm, or less than 1 μm).
1.20. Any preceding formulation, wherein the formulation further comprises the Compound of Formula II or the Compound of Formula III or a combination thereof.
1.21. Any preceding formulation, wherein the formulation comprises the Compound of Formula I in tosylate salt form, e.g., in mono-tosylate salt form or di-tosylate salt form, or a mixture thereof.
1.22. Formulation 1.21, wherein the formulation further comprises toluenesulfonic acid, e.g., wherein the formulation comprises the Compound of Formula I in tosylate salt form and toluenesulfonic acid in a ratio of 1:3 to 3:1, or 1:2 to 2:1 or 1:1.5 to 1.5:1, or about 1:1.
1.23. Any preceding formulation, wherein the Compound of Formula I is enriched in deuterium at one or more hydrogen atom positions, for example, wherein at any one or more hydrogen atom positions there is substantially greater than the natural level of incorporation of deuterium at such positions of the structure (e.g., greater than 0.1%, or greater than 0.5%, or greater than 1%, or greater than 5%).
1.24. Formulation 1.23, wherein the Compound of Formula I has greater than 50% incorporation of deuterium at any one or more hydrogen atom positions of the structure (i.e., greater than 50 atom % D), e.g., greater than 60%, or greater than 70%, or greater than 80%, or greater than 90% or greater than 95%, or greater than 96%, or greater than 97%, or greater than 98%, or greater than 99%.
1.25. Any of formulations 1 or 1.1 to 1.24, wherein all hydrogen atom positions of the Compound of Formula I are non-enriched in deuterium (i.e., every hydrogen atom position contains the natural abundance of deuterium or less than 0.1% deuterium).
1.26. Any of formulations 1 or 1.1 to 1.25, wherein the formulation systemically delivers a mean daily dose of the compound of Formula I of 0.1 to 5.0 mg per day (free base equivalent), e.g., 0.5 to 2.0 mg per day, or 0.1 to 0.5 mg per day, or 1.0 to 2.0 mg per day, or about 1.8 mg per day.
1.27. Any of formulations 1 or 1.1 to 1.26, wherein the formulation delivers the compound of Formula I at a rate sufficient to maintain a steady state maximum plasma concentration of the compound of Formula I (free base) of 5 to 50 ng/ml, e.g., 10 to 40 ng/mL, or 20 to 40 ng/ml (e.g., about 30 ng/ml) and/or sufficient to maintain a steady state 24-hour mean plasma concentration area under the curve (AUC) of the compound of Formula I (free base) of 5 to 100 ng-hr/mL, e.g., 20 to 90 ng-hr/mL, or 40 to 90 ng-hr/mL, or 70 to 90 ng hr/mL (e.g., about 80 ng-hr/mL).

In a second embodiment, the present disclosure provides a transdermal pharmaceutical formulation (Formulation 2) comprising 1-(4-fluoro-phenyl)-4-((6bR,10aS)-2,2-d₂-3-methyl-2,3,6b,9,10, 10a-hexahydro-1H, 7H-pyrido[3′,4′:4,5]pyrrolo[1,2,3-de]quinoxalin-8-yl)-butan-1-one (Compound of Formula II), in free base, co-crystal or salt form. The present disclosure further provides the following Formulations:
2.1. Formulation 2, wherein the formulation comprises the Compound of Formula II in free base form.
2.2. Formulation 2, wherein the formulation comprises the Compound of Formula II in salt form, e.g., in pharmaceutically acceptable salt form, optionally in amorphous solid or crystal salt form; or in co-crystal form, e.g., in nicotinamide or isonicotinamide co-crystal form.
2.3. Formulation 2.2, wherein the salt form is a tosylate, oxalate, cyclamate, 4-aminosalicylate, or hydrochloride salt form, optionally, wherein said salt form is a crystal salt form.
2.4. Any preceding formulation, wherein the formulation comprises from 1 to 1000 mg of the Compound of Formula II (free base equivalent), e.g., 1 to 750 mg, 1 to 500 mg, 1 to 300 mg, 1 to 200 mg, 10 to 200 mg, 50 to 200 mg, 100 to 200 mg, 100 to 300 mg, 200 to 300 mg, 200 to 500 mg, or 500 to 1000 mg.
2.5. Any preceding formulation, wherein the formulation comprises from 1 to 100 mg of the Compound of Formula II (free base equivalent), e.g., 10 to 70 mg, or 40 to 70 mg.
2.6. Any preceding formulation, wherein the formulation comprises from 1 to 80 mg of the Compound of Formula II (free base equivalent), e.g., from 1 to 50 mg, or from to 5 to 50 mg.
2.7. Any preceding formulation, wherein the composition further comprises one or more excipients, e.g., materials which stabilize the Compound of Formula II, and/or enhance absorption into the skin of the Compound of Formula II.
2.8. Formula 2.7, wherein the one or more excipients are selected from the group consisting of solvents, solubilizers, plasticizers, surfactants, binders, humectants (e.g., polyols), antioxidants, buffering agents (e.g., acids, bases and/or salts thereof), emollients, and thickening agents (e.g., gelling agents).
2.9. Formula 2.7, wherein the one or more excipients are selected from any of the following: alcohols (ethanol, isopropanol, propanol, glycerol, propylene glycol, butylene glycol, pentylene glycol, hexylene glycol, polyethylene glycol), non-alcoholic solvents (e.g., dimethylsulfoxide, dimethylformamide, acetonitrile), gums (e.g., acacia, guar, agar, xanthan, tragacanth, karaya, gellan), polysaccharides and polysaccharide derivatives (e.g., starches, dextrans, pectins, alginates, carrageenans, cellulose, cellulose derivatives (e.g., carboxymethyl cellulose, methylcellulose, hydroxyalkyl celluloses (e.g., hydroxyethyl cellulose, hydroxypropyl cellulose, hydroxypropyl methyl cellulose)), gelatins including non-gelling and gelling types (e.g., mammalian gelatins such as bovine gelatin, porcine gelatins, avian gelatins, fish gelatins (e.g., mixed high molecular weight and low molecular weight gelatins), synthetic polymers (e.g., polyvinylpyrrolidones, polyethylene oxide and/or polypropylene oxide polymers and copolymers (e.g., poloxamers, such as poloxamer 188), polyacrylate polymers (e.g., carbopols), polyamide polymers), sugars and sugar alcohols (e.g., dextrose, lactose, galactose, glucose, ribose, sucrose, trehalose, mannitol, maltitol, lactitol, sorbitol, xylitol, erythritol, galactitol, inositol) polypeptides/proteins, amino acids, inorganic or organic acids (e.g., citric acid, lactic acid, malic acid, mandelic acid, tropic acid, glycolic acid, gluconic acid, benzoic acid, toluenesulfonic acid, phosphoric acid, sulfuric acid, hydrochloric acid, tartaric acid, oxalic acid, cyclamic acid, ascorbic acid, methanesulfonic acid, benzenesulfonic acid, formic acid) and their salts (e.g., sodium, potassium, calcium, magnesium, lithium, ammonium salts of aforementioned acids) and esters (e.g., methyl or ethyl esters, or fatty acid esters), inorganic or organic bases (e.g., alkali metal or alkaline earth metal carbonates, bicarbonates, hydroxide, oxides), anionic surfactants (e.g., sodium lauryl sulfate, sodium laureth sulfate, sodium dodecylbenzenesulfonate, sodium lauroyl sarcosinate, sodium stearate), cationic surfactants (e.g., benzalkonium halides, cetylpyridinium halides, cetrimonium halides, benzethonium halides), zwitterionic surfactants (e.g., cocamidoalkyl betaines, such as cocamidopropyl betaine, or amine oxides, such as dimethyldodecylamine oxide and myristamine oxide), nonionic surfactants (e.g., fatty alcohol ethoxylates (e.g., polyethylene glycol polydodecyl ethers), sorbitan esters (e.g., sorbitan monolaurate, sorbitan monooleate, sorbitan monopalmitate, sorbitan monostearate, sorbitan tristearate), polyethoxylated sorbitan esters (e.g., polysorbate 20, polysorbate 40, polysorbate 60, polysorbate 80), fatty acid esters (e.g., lauryl lactate, isopropyl myristate, oleyl, oleate, methyl laurate, isopropyl palmitate, ethyl oleate), fatty alcohols (e.g., dodecanol, octyldecanol, lauryl alcohol), and antioxidants (e.g., ascorbic acid, citric acid, ascorbyl palmitate, sodium metabisulfite, BHT, BHA, TBHQ, propyl gallate, beta-carotene, tocopherols, tocotrienols).
2.10. Any of Formulations 2.7 to 2.9, wherein any one or more of said excipients are present in an amount of 0.1 to 80% by weight of the formulation, e.g., 0.1 to 60%, or 0.1 to 40%, or 0.1 to 20%, or 0.1 to 15% or 0.1 to 10%.
2.11. Any preceding formulation, wherein the formulation comprises an adhesive polymer, optionally in an amount from 25-80% by weight.
2.12. Formulation 2.11, wherein the adhesive polymer comprises a polymer selected from the group consisting of acrylate polymers or co-polymers (e.g., polymers in which at least one monomeric unit is an acrylic acid or ester, and in which one or more other monomeric units may be a non-acrylate monomer, e.g., a vinyl monomer), polyvinylpyrrolidones (e.g., linear or cross-linked), copolymers of maleic acid or a maleic ester with a vinyl ether (e.g., co-polymer of maleic acid or anhydride with methyl vinyl ether), cellulose derivatives (e.g., carboxymethyl cellulose), silicone polymers (e.g., dimethylsilicone), and mixtures thereof, such as, a mixture of a polyacrylate polymer and a silicone polymer.
2.13. Any preceding formulation, wherein the formulation is an ointment.
2.14. Any of formulations 2 or 2.1 to 2.11, wherein the formulation is a spray.
2.15. Any of formulations 2 or 2.1 to 2.11, wherein the formulation is a gel.
2.16. Any of formulations 2 or 2.1 to 2.11, wherein the formulation is comprised in a patch, e.g., an adhesive patch.
2.17. Formulation 2.16, wherein the patch comprises at least one drug reservoir layer and wherein the formulation is the drug reservoir layer.
2.18. Formulation 2.17, wherein the drug reservoir layer is adhesive.
2.19. Any preceding formulation wherein the Compound of Formula II is incorporated into the formulation as microparticles (e.g., particles having an average diameter of less than 50 μm, less than 30 μm, less than 10 μm, or less than 5 μm, or less than 1 μm).
2.20. Any preceding formulation, wherein the formulation further comprises the Compound of Formula I or the Compound of Formula III or a combination thereof.
2.21. Any preceding formulation, wherein the formulation comprises the Compound of Formula II in tosylate salt form, e.g., in mono-tosylate salt form or di-tosylate salt form, or a mixture thereof.
2.22. Formulation 2.21, wherein the formulation further comprises toluenesulfonic acid, e.g., wherein the formulation comprises the Compound of Formula II in tosylate salt form and toluenesulfonic acid in a ratio of 1:3 to 3:1, or 1:2 to 2:1 or 1:1.5 to 1.5:1, or about 1:1.
2.23. Any preceding formulation, wherein the Compound of Formula II is enriched in deuterium at one or more hydrogen atom positions, for example, wherein at any one or more hydrogen atom positions there is substantially greater than the natural level of incorporation of deuterium at such positions of the structure (e.g., greater than 0.1%, or greater than 0.5%, or greater than 1%, or greater than 5%).
2.24. Formulation 2.23, wherein the Compound of Formula II has greater than 50% incorporation of deuterium at any one or more hydrogen atom positions of the structure (i.e., greater than 50 atom % D), e.g., greater than 60%, or greater than 70%, or greater than 80%, or greater than 90% or greater than 95%, or greater than 96%, or greater than 97%, or greater than 98%, or greater than 99%.
2.25. Any of formulations 2 or 2.1 to 2.24, wherein all hydrogen atom positions of the Compound of Formula II are non-enriched in deuterium (i.e., every hydrogen atom position contains the natural abundance of deuterium or less than 0.1% deuterium;
2.26. Any of formulations 2 or 2.1 to 2.25, wherein the formulation systemically delivers a mean daily dose of the compound of Formula II of 0.1 to 5.0 mg per day (free base equivalent), e.g., 0.5 to 2.0 mg per day, or 0.1 to 0.5 mg per day, or 1.0 to 2.0 mg per day, or about 1.8 mg per day.
2.27. Any of formulations 2 or 2.1 to 2.26, wherein the formulation delivers the compound of Formula II at a rate sufficient to maintain a steady state maximum plasma concentration of the compound of Formula II (free base) of 5 to 50 ng/ml, e.g., 10 to 40 ng/mL, or 20 to 40 ng/ml (e.g., about 30 ng/mL) and/or sufficient to maintain a steady state 24-hour mean plasma concentration area under the curve (AUC) of the compound of Formula II (free base) of 5 to 100 ng-hr/mL, e.g., 20 to 90 ng-hr/mL, or 40 to 90 ng-hr/mL, or 70 to 90 ng hr/mL (e.g., about 80 ng-hr/mL).

In a third embodiment, the present disclosure provides a transdermal pharmaceutical formulation (Formulation 3) comprising 1-(4-fluoro-phenyl)-4-((6bR,10aS)-1,1,2,2-d₄-3-methyl-2,3,6b,9,10, 10a-hexahydro-1H,7H-pyrido[3′,4′:4,5]pyrrolo[1,2,3-de]quinoxalin-8-yl)-butan-1-one (Compound of Formula III), in free base, co-crystal or salt form. The present disclosure further provides the following Formulations:
3.1. Formulation 3, wherein the formulation comprises the Compound of Formula III in free base form.
3.2. Formulation 3, wherein the formulation comprises the Compound of Formula III in salt form, e.g., in pharmaceutically acceptable salt form, optionally in amorphous solid or crystal salt form; or in co-crystal form, e.g., in nicotinamide or isonicotinamide co-crystal form.
3.3. Formulation 3.2, wherein the salt form is a tosylate, oxalate, cyclamate, 4-aminosalicylate, or hydrochloride salt form, optionally, wherein said salt form is a crystal salt form.
3.4. Any preceding formulation, wherein the formulation comprises from 1 to 1000 mg of the Compound of Formula III (free base equivalent), e.g., 1 to 750 mg, 1 to 500 mg, 1 to 300 mg, 1 to 200 mg, 10 to 200 mg, 50 to 200 mg, 100 to 200 mg, 100 to 300 mg, 200 to 300 mg, 200 to 500 mg, or 500 to 1000 mg.
3.5. Any preceding formulation, wherein the formulation comprises from 1 to 100 mg of the Compound of Formula III (free base equivalent), e.g., 10 to 70 mg, or 40 to 70 mg.
3.6. Any preceding formulation, wherein the formulation comprises from 1 to 80 mg of the Compound of Formula III (free base equivalent), e.g., from 1 to 50 mg, or from to 5 to 50 mg.
3.7. Any preceding formulation, wherein the composition further comprises one or more excipients, e.g., materials which stabilize the Compound of Formula III, and/or enhance absorption into the skin of the Compound of Formula III.
3.8. Formula 3.7, wherein the one or more excipients are selected from the group consisting of solvents, solubilizers, plasticizers, surfactants, binders, humectants (e.g., polyols), antioxidants, buffering agents (e.g., acids, bases and/or salts thereof), emollients, and thickening agents (e.g., gelling agents).
3.9. Formula 3.7, wherein the one or more excipients are selected from any of the following: alcohols (ethanol, isopropanol, propanol, glycerol, propylene glycol, butylene glycol, pentylene glycol, hexylene glycol, polyethylene glycol), non-alcoholic solvents (e.g., dimethylsulfoxide, dimethylformamide, acetonitrile), gums (e.g., acacia, guar, agar, xanthan, tragacanth, karaya, gellan), polysaccharides and polysaccharide derivatives (e.g., starches, dextrans, pectins, alginates, carrageenans, cellulose, cellulose derivatives (e.g., carboxymethyl cellulose, methylcellulose, hydroxyalkyl celluloses (e.g., hydroxyethyl cellulose, hydroxypropyl cellulose, hydroxypropyl methyl cellulose)), gelatins including non-gelling and gelling types (e.g., mammalian gelatins such as bovine gelatin, porcine gelatins, avian gelatins, fish gelatins (e.g., mixed high molecular weight and low molecular weight gelatins), synthetic polymers (e.g., polyvinylpyrrolidones, polyethylene oxide and/or polypropylene oxide polymers and copolymers (e.g., poloxamers, such as poloxamer 188), polyacrylate polymers (e.g., carbopols), polyamide polymers), sugars and sugar alcohols (e.g., dextrose, lactose, galactose, glucose, ribose, sucrose, trehalose, mannitol, maltitol, lactitol, sorbitol, xylitol, erythritol, galactitol, inositol) polypeptides/proteins, amino acids, inorganic or organic acids (e.g., citric acid, lactic acid, malic acid, mandelic acid, tropic acid, glycolic acid, gluconic acid, benzoic acid, toluenesulfonic acid, phosphoric acid, sulfuric acid, hydrochloric acid, tartaric acid, oxalic acid, cyclamic acid, ascorbic acid, methanesulfonic acid, benzenesulfonic acid, formic acid) and their salts (e.g., sodium, potassium, calcium, magnesium, lithium, ammonium salts of aforementioned acids) and esters (e.g., methyl or ethyl esters, or fatty acid esters), inorganic or organic bases (e.g., alkali metal or alkaline earth metal carbonates, bicarbonates, hydroxide, oxides), anionic surfactants (e.g., sodium lauryl sulfate, sodium laureth sulfate, sodium dodecylbenzenesulfonate, sodium lauroyl sarcosinate, sodium stearate), cationic surfactants (e.g., benzalkonium halides, cetylpyridinium halides, cetrimonium halides, benzethonium halides), zwitterionic surfactants (e.g., cocamidoalkyl betaines, such as cocamidopropyl betaine, or amine oxides, such as dimethyldodecylamine oxide and myristamine oxide), nonionic surfactants (e.g., fatty alcohol ethoxylates (e.g., polyethylene glycol polydodecyl ethers), sorbitan esters (e.g., sorbitan monolaurate, sorbitan monooleate, sorbitan monopalmitate, sorbitan monostearate, sorbitan tristearate), polyethoxylated sorbitan esters (e.g., polysorbate 20, polysorbate 40, polysorbate 60, polysorbate 80), fatty acid esters (e.g., lauryl lactate, isopropyl myristate, oleyl oleate, methyl laurate, isopropyl palmitate, ethyl oleate), fatty alcohols (e.g., dodecanol, octyldecanol, lauryl alcohol), and antioxidants (e.g., ascorbic acid, citric acid, ascorbyl palmitate, sodium metabisulfite, BHT, BHA, TBHQ, propyl gallate, beta-carotene, tocopherols, tocotrienols).
3.10. Any of Formulations 3.7 to 3.9, wherein any one or more of said excipients are present in an amount of 0.1 to 80% by weight of the formulation, e.g., 0.1 to 60%, or 0.1 to 40%, or 0.1 to 20%, or 0.1 to 15% or 0.1 to 10%.
3.11. Any preceding formulation, wherein the formulation comprises an adhesive polymer, optionally in an amount from 25-80% by weight.
3.12. Formulation 3.11, wherein the adhesive polymer comprises a polymer selected from the group consisting of acrylate polymers or co-polymers (e.g., polymers in which at least one monomeric unit is an acrylic acid or ester, and in which one or more other monomeric units may be a non-acrylate monomer, e.g., a vinyl monomer); polyvinylpyrrolidones (e.g., linear or cross-linked), copolymers of maleic acid or a maleic ester with a vinyl ether (e.g., co-polymer of maleic acid or anhydride with methyl vinyl ether), cellulose derivatives (e.g., carboxymethyl cellulose), silicone polymers (e.g., dimethylsilicone), and mixtures thereof, such as, a mixture of a polyacrylate polymer and a silicone polymer.
3.13. Any preceding formulation, wherein the formulation is an ointment.
3.14. Any of formulations 3 or 3.1 to 3.11, wherein the formulation is a spray.
3.15. Any of formulations 3 or 3.1 to 3.11, wherein the formulation is a gel.
3.16. Any of formulations 3 or 3.1 to 3.11, wherein the formulation is comprised in a patch, e.g., an adhesive patch.
3.17. Formulation 3.16, wherein the patch comprises at least one drug reservoir layer and wherein the formulation is the drug reservoir layer.
3.18. Formulation 3.17, wherein the drug reservoir layer is adhesive.
3.19. Any preceding formulation wherein the Compound of Formula III is incorporated into the formulation as microparticles (e.g., particles having an average diameter of less than 50 μm, less than 30 μm, less than 10 μm, or less than 5 μm, or less than 1 μm).
3.20. Any preceding formulation, wherein the formulation further comprises the Compound of Formula I or the Compound of Formula II or a combination thereof.
3.21. Any preceding formulation, wherein the formulation comprises the Compound of Formula III in tosylate salt form, e.g., in mono-tosylate salt form or di-tosylate salt form, or a mixture thereof.
3.22. Formulation 3.21, wherein the formulation further comprises toluenesulfonic acid, e.g., wherein the formulation comprises the Compound of Formula III in tosylate salt form and toluenesulfonic acid in a ratio of 1:3 to 3:1, or 1:2 to 2:1 or 1:1.5 to 1.5:1, or about 1:1.
3.23. Any preceding formulation, wherein the Compound of Formula III is enriched in deuterium at one or more hydrogen atom positions, for example, wherein at any one or more hydrogen atom positions there is substantially greater than the natural level of incorporation of deuterium at such positions of the structure (e.g., greater than 0.1%, or greater than 0.5%, or greater than 1%, or greater than 5%).
3.24. Formulation 3.23, wherein the Compound of Formula III has greater than 50% incorporation of deuterium at any one or more hydrogen atom positions of the structure (i.e., greater than 50 atom % D), e.g., greater than 60%, or greater than 70%, or greater than 80%, or greater than 90% or greater than 95%, or greater than 96%, or greater than 97%, or greater than 98%, or greater than 99%.
3.25. Any of formulations 3 or 3.1 to 3.24, wherein all hydrogen atom positions of the Compound of Formula III are non-enriched in deuterium (i.e., every hydrogen atom position contains the natural abundance of deuterium or less than 0.1% deuterium).
3.26. Any of formulations 3 or 3.1 to 3.25, wherein the formulation systemically delivers a mean daily dose of the compound of Formula III of 0.1 to 5.0 mg per day (free base equivalent), e.g., 0.5 to 2.0 mg per day, or 0.1 to 0.5 mg per day, or 1.0 to 2.0 mg per day, or about 1.8 mg per day.
3.27. Any of formulations 3 or 3.1 to 3.26, wherein the formulation delivers the compound of Formula III at a rate sufficient to maintain a steady state maximum plasma concentration of the compound of Formula III (free base) of 5 to 50 ng/ml, e.g., 10 to 40 ng/mL, or 20 to 40 ng/ml (e.g., about 30 ng/mL) and/or sufficient to maintain a steady state 24-hour mean plasma concentration area under the curve (AUC) of the compound of Formula III (free base) of 5 to 100 ng-hr/mL, e.g., 20 to 90 ng-hr/mL, or 40 to 90 ng-hr/mL, or 70 to 90 ng hr/mL (e.g., about 80 ng-hr/mL).

In a fourth embodiment, the present disclosure provides a transdermal pharmaceutical device (Device 1) comprising any of Formulation 1 et seq, Formula 2 et seq, or Formula 3 et seq. In particular embodiments, the present disclosure further provides:
1.1. Device 1, wherein the device comprises at least two layers, wherein at least one layer is a drug reservoir layer (e.g., a layer comprising the compound of Formula I, or Formula II, or Formula III), and at least one layer is a backing layer (e.g., a layer which is an inert layer and which is substantially free of and substantially impermeable to the compounds of Formula I, Formula II and Formula III).
1.2. Device 1.1, wherein the drug reservoir layer comprises or consists of Formulation 1 or any of 1.1-1.27.
1.3. Device 1.1, wherein the drug reservoir layer comprises or consists of Formulation 2 or any of 2.1-2.27.
1.4. Device 1.1, wherein the drug reservoir layer comprises or consists of Formulation 3 or any of 3.1-3.27.
1.5. Any preceding Device, wherein the drug reservoir layer comprises an adhesive polymer, as defined hereinabove, and wherein the drug reservoir layer is the skin-contacting layer of the device.
1.6. Device 1.5, wherein the drug reservoir layer comprises the adhesive polymer in an amount from 25-80% by weight, e.g., from 30-65% by weight, or from 40 to 60% by weight.
1.7. Device 1 or any of 1.1-1.4, wherein the Device further comprises a third layer, separate from the drug reservoir layer, which third layer is the skin-contacting layer and which layer comprises an adhesive polymer, as defined hereinabove, and which layer is substantially free of the compounds of Formula I, Formula II and Formula III prior to use of the device.
1.8. Device 1.7, wherein the skin-contacting layer comprises the adhesive polymer in an amount from 25-90% by weight, e.g., from 50-90% by weight, or from 65 to 90% by weight.
1.9. Device 1.7 or 1.8, wherein the device further comprises an intermediate layer disposed between the skin contact layer and the drug reservoir layer, which layer is a rate-control layer optionally comprising any of the excipients set forth hereinabove.
1.10. Device 1.9, wherein the intermediate layer is a microporous polymer layer, e.g., wherein the polymer layer comprises polyethylene, polypropylene, polyurethane, polyester, vinyl acetate polymer or copolymer, or combinations thereof.
1.11. Any preceding device, wherein the drug reservoir layer and/or the skin-contacting layer comprise one or more permeation enhancing excipients, for example, a fatty acid ester (e.g., lauryl lactate), an alcohol (e.g., propylene glycol), an amine oxide (e.g., dimethyldodecyl amine oxide), and/or a carboxylic acid (e.g., an alpha-hydroxy acid, e.g. lactic acid).
1.12. Any preceding device wherein the drug reservoir layer comprises the Compound of Formula I, or the compound of Formula II, or the compound of Formula III in free base form.
1.13. Any preceding device wherein the drug reservoir layer comprises the Compound of Formula I, or the compound of Formula II, or the compound of Formula III in pharmaceutically acceptable salt form (e.g., tosylate salt form) in admixture with at least one organic or inorganic base, e.g., in order to slowly generate the free base form of said Compound.
1.14. Any preceding device, wherein the backing layer is flexible or partially flexible.
1.15. Any preceding device, wherein the backing layer is comprised of one or more of a film, a non-woven fabric, a woven fabric, a laminate, or combinations thereof.
1.16. Any preceding device, wherein the backing layer comprises one or more polymers selected from polyesters, polyamides, polyvinylidene chloride, polyvinyl chloride, polyethylene, polypropylene, polyurethane, polyether amides, polyvinyl acetate, nylons.
1.17. Any preceding device further comprising a release liner which is at least partially in contact with the skin-contacting layer or drug reservoir layer and which protects the adhesive elements of the device prior to use and prevents migration of any components of the device out of the device prior to use.
1.18. Device 1.17, wherein the release liner is an impermeable polymer film.
1.19. Any preceding device, wherein the device comprises from 1 to 1000 mg of the Compound of Formula I or the Compound of Formula II or the Compound of Formula III (free base equivalent), e.g., 1 to 750 mg, 1 to 500 mg, 1 to 300 mg, 1 to 200 mg, 10 to 200 mg, 50 to 200 mg, 100 to 200 mg, 100 to 300 mg, 200 to 300 mg, 200 to 500 mg, or 500 to 1000 mg.
1.20. Any preceding device, wherein the device is formulated to deliver the Compound of Formula I or the Compound of Formula II or the Compound of Formula III to a patient over a period of time from 1 to 30 days, e.g., from 1 to 20 days, or from 1 to 15 days, or from 1 to 10 days, or from 1 to 7 days, or from 1 to 5 days, or from 1 to 3 days.
1.21. Any preceding device, wherein the device is formulated to deliver the Compound of Formula I or the Compound of Formula II or the Compound of Formula III to a patient at an average daily dosage of from 1 to 60 mg per day (free base equivalent), e.g., 1 to 40 mg per day, or 1 to 30 mg per day, or 1 to 20 mg per day, or 1 to 15 mg per day, or 1 to 10 mg per day.
1.22. Any preceding device, wherein the device provides an in vitro flux of the Compound of Formula I or the Compound of Formula II or the Compound of Formula III of between 10 and 500 μg/hr/cm² (free base equivalent).
1.23. Any preceding device, wherein the device systemically delivers a mean daily dose of the compound of Formula I or the Compound of Formula II or the Compound of Formula III of 0.1 to 5.0 mg per day (free base equivalent), e.g., 0.5 to 2.0 mg per day, or 0.1 to 0.5 mg per day, or 1.0 to 2.0 mg per day, or about 1.8 mg per day.
1.24 Any preceding device, wherein the device delivers the compound of Formula I or the Compound of Formula II or the Compound of Formula III at a rate sufficient to maintain a steady state maximum plasma concentration of the compound (free base) of 5 to 50 ng/mL, e.g., 10 to 40 ng/ml, or 20 to 40 ng/mL (e.g., about 30 ng/ml) and/or sufficient to maintain a steady state 24-hour mean plasma concentration area under the curve (AUC) of the compound (free base) of 5 to 100 ng-hr/mL, e.g., 20 to 90 ng-hr/mL, or 40 to 90 ng-hr/mL, or 70 to 90 ng hr/mL (e.g., about 80 ng-hr/mL).

As used herein, the term “transdermal formulation” or “transdermal delivery” does not embrace “trans-mucosal” routes of administration, such as sublingual, oral, buccal, vaginal intranasal and pulmonary. Transdermal delivery refers to the transport of a drug substance through the skin, e.g., keratinized epithelium, for absorption in the capillaries within or below the dermal layer of the skin for systemic delivery of the drug substance.

As used herein, “deuteration” refers to the substitution of a hydrogen (protium, ¹H) atom in a chemical structure with a deuterium atom (²H). A hydrogen atom position of a structure is considered substituted with deuterium when the abundance of deuterium at that position is enriched. The natural abundance of deuterium is about 0.02%, so a compound is “enriched” with deuterium at a specific position when the frequency of incorporation of deuterium at that position exceeds 0.02%. Therefore, in any embodiment of a deuterated compound provided herein, any one or more hydrogen atoms may be enriched with deuterium at a level of greater than 0.1%, or greater than 0.5%, or greater than 1%, or greater than 5%, such as, greater than 50%, or greater than 60%, or greater than 70%, or greater than 80%, or greater than 90% or greater than 95%, or greater than 96%, or greater than 97%, or greater than 98%, or greater than 99%.

In all aspects and embodiments of the present disclosure comprising the Compound of Formula I, said Compound of Formula I may optionally be deuterated at one or more hydrogen atom positions.

In all aspects and embodiments of the present disclosure comprising the Compound of Formula II, said Compound of Formula II is enriched only at the designated positions (2,2-d₂).

In all aspects and embodiments of the present disclosure comprising the Compound of Formula III, said Compound of Formula III is enriched only at the designated positions (1,1,2,2-d₄).

In some embodiments, the formulations may further comprise one or more anti-oxidants, for example, tocopherol, butylated hydroxytoluene (BHT), propyl gallate (OPG), citric acid, and/or ascorbic acid, or the like. In some embodiments, the formulations do not comprise any anti-oxidant compounds. The inclusion of an anti-oxidant may further improve the chemical stability of the formulations by preventing oxidative chemical degradation of the active ingredient.

In another aspect, the present disclosure provides Formulation 1, et seq., Formulation 2, et seq., Formulation 3, et seq., or Device 1, et seq., for use in treating a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (including major depressive disorder (MDD)), anxiety, psychosis, schizophrenia, obsessive-compulsive disorder, sexual disorders, migraine, attention deficit disorder, attention deficit hyperactivity disorder, sleep disorders, conditions associated with cephalic pain, social phobias, dementia (including Alzheimer's Disease and Parkinson's dementia), gastrointestinal disorders such as dysfunction of gastrointestinal tract motility and obesity, or bipolar disorder (e.g., bipolar depression).

In another embodiment, the invention provides a method (Method 1) for the prophylaxis or treatment of a disease or abnormal condition involving or mediated by the 5-HT_{2A} receptor, serotonin transporter (SERT), and/or dopamine D₁/D₂ receptor signaling pathways, in a patient in need thereof, comprising administering to the patient by a transdermal route, a therapeutically effective amount of the Compound of Formula I and/or the Compound of Formula II and/or the Compound of Formula III. Further embodiments of Method 1 include:
1.1 Method 1, wherein the Compound of Formula I and/or the Compound of Formula II and/or the Compound of Formula III is administered via a transdermal route (e.g., administered using a transdermal delivery device, such as a patch, gel, ointment, or spray).
1.2 Method 1.1, wherein the Compound of Formula I and/or the Compound of Formula II and/or the Compound of Formula III is administered in the form of a composition selected from any of Formulation 1, et seq., or Formulation 2, et seq. or Formulation 3, et seq.
1.3 Method 1.1 or 1.2 wherein the Compound of Formula I and/or the Compound of Formula II and/or the Compound of Formula III is administered via Device 1, et seq.
1.4 Method 1 or any of 1.1, et seq., wherein the disease or condition is selected from obesity, anorexia, bulimia, depression (including major depressive disorder (MDD)), anxiety, psychosis, schizophrenia, obsessive-compulsive disorder, sexual disorders, migraine, attention deficit disorder, attention deficit hyperactivity disorder, sleep disorders, conditions associated with cephalic pain, social phobias, dementia (including Alzheimer's Disease and Parkinson's dementia), gastrointestinal disorders such as dysfunction of gastrointestinal tract motility and obesity, or bipolar disorder (e.g., bipolar depression).
1.5 Method 1 or any of 1.1, et seq., wherein the daily dosage administered via the transdermal route (e.g., via Device 1, et seq.) provides from 1 to 50% of the dosage administered for the same condition by the oral route, for example, 1 to 40%, or from 5 to 25%, or from 5 to 15%, or from 1-5%, or from 2.5 to 7.5% of the daily oral dosage (due to reduced first pass and GI metabolism).
1.6 Method 1 or any of 1.1, et seq., wherein the method comprises transdermal application of the transdermal delivery device once every 30 days or less, e.g., once every 20 days or less, or once every 15 days or less, or once every 10 days or less, or once every 7 days or less, or once every 5 days or less, or once every 3 days or less.
1.7 Method 1 or any of 1.1, et seq., wherein the method comprises delivery of the Compound of Formula I or the Compound of Formula II or the Compound of Formula III to the patient at an average daily dosage of from 5 to 60 mg per day (free base equivalent), e.g., 5 to 40 mg per day, or 5 to 30 mg per day, or 5 to 20 mg per day, or 5 to 15 mg per day, or 5 to 10 mg per day, or 1 to 5 mg per day, or 1 to 3 mg per day or 2 to 3 mg per day, or about 1.8 mg per day, or 0.1 to 2 mg per day, or 0.1 to 1 mg per day, or 0.1 to 0.7 mg per day, or about 0.6 mg per day.

A Compound of Formula I or a Compound of Formula II and/or a Compound of Formula III, for use in Method 1 or any of Method 1.1 et seq.

A pharmaceutical composition selected from any of Formulation 1, et seq., Formulation 2, et seq., or Formulation 3, et seq., or Device 1, et seq., for use in Method 1 or any of Method 1.1 et seq.

Use of a Compound of Formula I or a Compound of Formula II or a Compound of

Formula III in the manufacture of a medicament for transdermal administration, e.g., a medicament in accordance with any of Formulation 1, et seq., Formulation 2, et seq., or

Formulation 3, et seq., or Device 1, et seq., for use in a method of treatment according to Method 1 or any of Method 1.1 et seq.

Methods of synthesizing the Compounds of Formula I and the Compounds of Formula II and III are known in art, and include the methods disclosed in U.S. Pat. Nos. 6,548,493; 7,238,690; 6,552,017; 6,713,471; 7,183,282; 7,081,455; 8,309,722; U.S. RE39680, and U.S. RE39679, and US 2017/183350, the contents of each of which are incorporated by reference in their entirety. Salts of the Compounds of the Invention may also be prepared as similarly described in U.S. Pat. Nos. 6,548,493; 7,238,690; 6,552,017; 6,713,471; 7,183,282; 8,648,077; U.S. RE39680; and U.S. RE39679, the contents of each of which are incorporated by reference in their entirety.

Isolation or purification of the diastereomers of the Compounds of the Invention may be achieved by conventional methods known in the art, e.g., column purification, preparative thin layer chromatography, preparative HPLC, crystallization, trituration, simulated moving beds and the like.

The pharmaceutically acceptable salts of the Compounds of Formula I, II and III can be synthesized from the parent compound which contains a basic or acidic moiety by conventional chemical methods. Generally, such salts can be prepared by reacting the free base forms of these compounds with a stoichiometric amount of the appropriate acid in water or in an organic solvent, or in a mixture of the two; generally, non-aqueous media like ether, ethyl acetate, ethanol, isopropanol, or acetonitrile are preferred. Further details for the preparation of these salts, e.g., toluenesulfonic salt in amorphous or crystal form, may be found in U.S. Pat. Nos. 8,309,722 and/or 8,648,077.

Transdermal dosage forms are known to those skilled in the art, and general procedures by which these dosage forms can be prepared have been described. Examples of such delivery systems include those disclosed in U.S. Pat. No. 9,993,466 to Lee et al., U.S. Pat. No. 9,913,840 to Jain et al., U.S. Pat. No. 9,693,970 to Mo, U.S. Pat. No. 9,585,862 to Hwang et al., and U.S. Pat. No. 7,858,114 to Ito. The contents of each of these references is incorporated by reference herein in their entireties.

### EXAMPLES

### Example 1A: Comparison of Pharmacokinetics between Subcutaneous (SC) and Sublingual (SL) Dosing in Dogs

In vivo absorption and distribution of the Compound of Formula I and the Compound of Formula II, both in their tosylate salt forms, is compared between subcutaneous and sublingual administration in non-cross over sequential studies in dogs.

SC Administration: Six male beagle dogs between 2 and 5 years of age are randomized in two groups of three dogs each. Dogs in group 1 are administered the Compound of Formula I at a dose of 1 mg/kg (free base equivalent) in a 0.5% methylcellulose/distilled water vehicle. Dogs in group 2 are administered the Compound of Formula II at a dose of 1 mg/kg (free base equivalent) in a 0.5% methylcellulose/distilled water vehicle. Administration is subcutaneous in the intrascapular region via a 22 or 23 gauge needle. Whole blood samples are collected via the dog's cephalic vein pre-dose, and at post-dose time-points 5, 15 and 30 minutes, 1, 2, 4, 6, 8 and 24 hours. Following a minimum 7-day washout period, the dogs are transferred to the sublingual portion of the study.

SL Administration: The dogs of group 1 are administered the Compound of Formula I at a dose of 1 mg/kg (free base equivalent) in a 0.5% methylcellulose/distilled water vehicle. Dogs in group 2 are administered the Compound of Formula II at a dose of 1 mg/kg (free base equivalent) in a 0.5% methylcellulose/distilled water vehicle. The animals are anesthetized prior to administration of the dose using propofol (6 mg/kg) and anesthesia is maintained for 30 minutes using 3-4.5% isoflurane. Administration is sublingual and the dosage is applied for 30 minutes, then wiped off using unwoven gauze. Whole blood samples are collected via the dog's cephalic vein pre-dose, and at post-dose time-points 5, 15 and 30 minutes, 1, 2, 4, 6, 8, 24, 36 and 48 hours.

All blood samples are processed to plasma and analyzed for drug concentrations using liquid chromatography-tandem mass spectrometry (LC-MS/MS). Area under the curve (AUC) of parent and metabolites based on plasma versus time data are calculated using Prism 5.04 software (GraphPad Software, Inc.).

The results are summarized in Table 1A below (AUC is shown for 0-24 hours):

| | Test Compound (1 mg/kg): | Formula I | Formula II |
|---|---|---|---|
| SL | AUC (ng-hr/mL) | 734 | 1262 |
| | Cmax (ng/mL) | 259 | 562 |
| | Tmax (hr) | 1.0 | 1.0 |
| SC | AUC (ng-hr/mL) | 813 | 785 |
| | Cmax (ng/mL) | 110 | 79 |
| | Tmax (hr) | 1.0 | 1.0 |

The results demonstrate that both SC and SL dosing results in high plasma concentration and high plasma AUC. SL dosing also results in significantly higher maximal plasma concentration of drug (Cmax) compared to SC dosing for both compounds. For the Compound of Formula II, SL dosing also results in a significantly higher overall AUC, while the AUC for the Compound of Formula I shows comparable AUC between SC and SL dosing. Time to maximum plasma concentration is also the same between SC and SL dosing for both compounds.

### Example 1B: Comparison of Pharmacokinetics between Subcutaneous and Sublingual Dosing in Dogs with Metabolite Analysis

A second study is performed substantially as described for Example 1A, except that plasma samples are analyzed for the parent (administered) compounds, as well as for the major known metabolites. After administration of either the Compound of Formula I or the Compound of Formula II, the major circulating species are found to be the parent and the N-desmethyl metabolite. The results are summarized in Table 1B below (AUC is shown for 0-24 hours):

| | Test Compound (1 mg/kg): | Formula I | Formula II |
|---|---|---|---|
| SL | AUC (ng-hr/mL)-Parent | 507 | 1262 |
| | Cmax (ng/mL)-Parent | 179 | 562 |
| | Tmax (hr)-Parent | 1.0 | 1.0 |
| | AUC (ng-hr/mL)-Des-methyl | 23 | 104 |
| | Cmax (ng/mL)-Des-methyl | 4.0 | 27 |
| | Tmax (hr)-Des-methyl | 1.0 | 1.0 |
| SC | AUC (ng-hr/mL)-Parent | 560 | 785 |
| | Cmax (ng/mL)-Parent | 76 | 79 |
| | Tmax (hr)-Parent | 1.0 | 1.0 |
| | AUC (ng-hr/mL)-Des-methyl | 20 | 49 |
| | Cmax (ng/mL)-Des-methyl | 4.0 | 1.0 |
| | Tmax (hr)-Des-methyl | 1.7 | 1.0 |

These results further demonstrate that both SC and SL dosing results in high plasma concentration and high plasma AUC for the administered compounds. SL dosing also results in higher maximal plasma concentration of drug (Cmax) compared to SC dosing for both compounds. In addition, the results show that both SC and SL dosing results in very low rates of metabolite formation, indicating that these routes effectively bypass the primary site of metabolic degradation of these compounds (hepatic metabolism). The Compound of Formula I circulated in plasma at an AUC approximately 22 times higher for parent than metabolite after SL dosing, and 27 times higher for SC dosing. In contrast, when administered orally, it has been found that the Compound of Formula I and its desmethyl metabolite circulate in plasma with AUC's of about 1:1 or less. Similarly, results are shown for the Compound of Formula II (parent/metabolite ratio of about 12 for SL and about 16 for SC).

### Example 2: Pharmacokinetics of Oral Dosing in Dogs

In contrast to the subcutaneous and sublingual pharmacokinetics presented in Example 1, this Example demonstrates that oral administration results in substantially lower systemic exposure to drug, due to the high extent of hepatic first-pass metabolism. This is true even using doses from 2.5 times to 15 times higher than the dose used in the SC and SL study.

As part of a larger long-term toxicology study, 20 male and female beagle dogs are administered either control, or the Compound of Formula I, tosylate salt form, at a dose of 2.5, 5, 10, or 15 mg/kg. For control, the dogs are administered empty capsules. For the Compound of Formula I, the dogs are administered normal-release oral capsules. Blood samples are obtained at 0.25 hours, 0.5 hours, 1 hour, 4 hours, 8 hour and 24 hours. The blood samples are processed to plasma and analyzed for concentration of the Compound of Formula I by high-performance liquid chromatography-mass spectrometry. Cmax, Tmax and area-under-the-curve (AUC, 0-24 hours) are calculated using Phoenix WinNonlin software. The results are shown in Table 1 below:

| | Dosing: | | | |
|---|---|---|---|---|
| | 2.5 mg/kg | 5 mg/kg | 10 mg/kg | 15 mg/kg |
| AUC (ng-hr/mL) | 40.05 | 52.45 | 142.5 | 248 |
| Cmax (ng/mL) | 5.51 | 7.72 | 29.0 | 44.5 |
| Tmax (hr) | 0.469 | 0.875 | 0.813 | 1.63 |

The Compound of Formula I shows dose-dependent oral absorption, and that the plasma concentration achieved with even the highest-oral dose is far below the plasma concentration and AUC achieved from sublingual and subcutaneous administration as shown in Example 1. Sublingual and subcutaneous dosing of the Compound of Formula I at only 1.0 mg/kg results in 24-hour AUC that is more than 18× higher than oral dosing at 2.5 mg/kg. Sublingual and subcutaneous doing even provide 24-hour AUC about three times higher at 1.0 mg/kg than oral dosing at 15 mg/kg.

These results taken together clearly demonstrate the large loss in exposure resulting from hepatic first pass metabolism, and the unexpectedly high improvement in exposures that are achieved from using transdermal dosing systems.

### Example 3: Pharmacokinetics of SC and IV Dosing in Monkeys

A study is also performed in monkeys to determine the plasma concentrations of both the Compound of Formula I and its major metabolites after both SL and SC dosing at 0.5 mg/kg. Six Cynomolgus monkeys are divided into two groups for IV and SC dosing with the Compound of Formula I. The animals are dosed in a fasting state. Blood samples are collected pre-dose, and at 5 minutes, 15 minutes, 1, 2, 4, 6, 8, 24, 36 and 48 hours post-dose. All blood samples are processed to plasma and later analyzed by liquid chromatography-tandem mass spectrometry. Samples are tested for the concentration of the Compound of Formula I, as well as for its five major known metabolites. PK parameters are calculated using PK Solutions 2.0 software (Summit Research Services, Colorado, US).

The results show that bioavailability (based on a comparison of IV to SC pharmacokinetics) is about 74% for the Compound of Formula I or SC administration. Most tested metabolites are found to present at levels below the limit of quantitation. Only the des-methyl metabolite and the amide metabolite (oxidation of the methylene adjacent to the N-methyl group to a carbonyl) are found to be present in significant concentration. The concentrations of both metabolites are found to be lower after SC dosing than after IV dosing. The results are shown in Table 3 below:

| | | Parent | Des-methyl | Amide |
|---|---|---|---|---|
| | Analyte: | Compound | metabolite | Metabolite |
| IV | AUC (ng-hr/mL) | 297 | 9.2 | 20 |
| | Cmax (ng/mL) | 94 | 1.5 | 1.9 |
| | Tmax (hr) | 0.083 | 1.0 | 4.0 |
| SC | AUC (ng-hr/mL) | 220 | 2.3 | 3.3 |
| | Cmax (ng/mL) | 23 | 0.3 | 0.3 |
| | Tmax (hr) | 2.0 | 2.0 | 6.0 |

The results show that SC administration in monkeys results in substantially lower levels of formation of the major metabolites compared to IV administration.

All of the foregoing results demonstrate the effectiveness of administering the compounds of the present disclosure in a manner which avoids first-pass metabolism.

### Example 4: In-Vitro Permeation Studies

Dual-layer transdermal patches are prepared which comprise an adhesive drug reservoir layer and a backing layer, protected by a release liner. Permeation studies are performed by removing the release liner from a sample patch, and immediately applying the patch to the stratum corneum layer of human cadaver skin fixed to the donor chamber side of a modified Franz diffusion cell. Mesh is applied over the patch to ensure that it remains adhered to the skin during the length of the experiment. The bottom chamber of the cell contains a receptor solution that is sampled at various time points and analyzed for lumateperone content. The cell includes a magnetic mixer bar to ensure thorough mixing. These measurements are used over the course of the experiment to calculate drug flux and cumulative delivery. All experiments use Franz cells with 2 square centimeter diffusional areas and 5.0 mL receptor solution capacities. The transdermal patches are 1 square centimeter in size. Temperature is maintained at 32° C., and humidity is ambient. The receptor solutions consist of 20% v/v polyethylene glycol 400 in pH 7.4 phosphate buffer. Solution samples are analyzed by HPLC.

Study 1. Patches are prepared comprising a two-component drug reservoir layer, consisting of 2.5% w/w lumateperone free base in a single excipient. 25 excipients are tested Samples from the receptor solutions are taken at 3, 6, 12 and 24 hours after application. Cumulative permeation results are shown in the table below:

| | Cum. |
|---|---|
| | Permation |
| Excipient | (μg) |
| Lauryl lactate | 90 |
| Isopropyl myristate | 68 |
| Oleayl Oleate | 61 |
| Methyl laurate | 57 |
| Isopropyl palmitate | 48 |
| Ethyl oleate | 44 |
| Octyldodecanol | 38 |
| Propylene glycol | 25 |
| Others | <15 |

The results show that of the 25 excipients selected, eight significantly outperform the other sixteen in promoting. The sixteen other excipients, which all resulted in cumulative permeations of 2-15 μg, are: butylene glycol, diethylene glycol monomethyl ether, diisopropyl adipate, dipropylene glycol, glyceryl monooleate, glycerol, isostearic acid, laureth-4, levulinic acid, octisalate, oleic acid, oleyl alcohol, propylene glycol monolaurate, sorbitan monooleate, and triacetin.

Study 2. Patches are prepared comprising a three-component drug reservoir layer, consisting of 10% w/w lumateperone free base and 20% w/w excipient in a polyacrylate vehicle. The eight preferred excipients from Study 1 are tested, along with a control containing 10% w/w lumateperone free base in polyacrylate vehicle. Samples from the receptor solutions are taken at 6, 24, 48 and 72 hours after application. A significant difference is observed in the presence of excipient compared with the absence of excipient. Optimal results are obtained with lauryl lactate excipient, resulting in 114 μg drug delivery over 72 hours, followed by propylene glycol and methyl laurate (50-70 μg), with the remaining excipients showing results similar to control (all 25-40 μg).

Study 3. Patches are prepared comprising a four-component drug reservoir layer, consisting of 10% w/w lumateperone free base and 0-10% w/w lauryl lactate excipient in a mixed polyacrylate adhesive and silicone adhesive vehicle. Samples from the receptor solutions are taken at 6, 24, 48 and 72 hours after application. As expected, the samples with 0% excipient produce the lowest permeation values, all less than 30 μg at 72 hours, while the samples with 10% excipient produce the highest permeation values, all greater than 70 μg at 72 hours. Optimal results are shown for the combination of 10% lauryl lactate, 40% acrylate adhesive and 40% silicone adhesive.

Further studies. Further studies are performed which examine an additional 20 excipients for their effect on permeation enhancement. Of these additional excipients, only methyl caprate and Labrafac PG® (propylene glycol dicaprate/dicaprylate) are found to significantly enhance permeation.

Further studies also evaluate 10% w/w lumateperone in various excipient mixtures in a polyacrylate/silicone adhesive vehicle. Each mixture includes a combination of excipients selected from lauryl lactate (LL), propylene glycol (PG), methyl laurate (ML), and propylene glycol dicaprate/dicaprylate (PGDD). It is found that there is an unexpected synergistic effect on permeation resulting from the combination of lauryl lactate with propylene glycol, as shown in the summary below:

| | Cumulative 72 |
|---|---|
| Excipient Mixture (s) | hr Permation (μg) |
| 10% LL, 1% PG | 170-180 |
| 5% LL, 5% PG, 5% ML, | 140-170 |
| 5% PGDD; 20% LL; | |
| 10% LL, 10% ML; | |
| 10% PG, 10% ML; | 80-110 |
| 10% PG, 10% PGDD; | |
| 20% ML; | |
| 20% PG | |
| 10% ML, 10% PGDD; | 40-60 |
| 20% PGDD | |

Further studies also evaluate dimethyldodecylamine oxide (DDAO) as an enhancing excipient. It is unexpectedly found that add just 2 wt % DDAO to the formulation comprising 20% lumateperone, 15% lauryl lactate, and 10% propylene glycol results in nearly a doubling of permeation at 72 hours, from about 300 μg to more than 550 μg.

It was further unexpectedly found that lactic acid enhances permeation. This is believed to result from its interfere with cohesion of the adhesive matrix. Comparative studies are further performed and show that lactic acid produces more favorable enhancement of permeation than the similar acids glycolic acid, mandelic acid and tropic acid.

Finally, six final drug reservoir layer formulations each having 20 wt % lumateperone are tested in a swine 3-day irritation and drug delivery study. Patches are also tested for stability in a 2-month aging study. For the swine studies, approximately 2″×2″ patches are placed adjacent in series on the animal's back, with the edges taped down with medical adhesive tape. An additional adhesive-only control is used for comparison of irritation results. Drug delivery is determined by measuring the amount of drug remaining in the patch and on the surface of the skin (collected by swabbing) compared to the amount of drug originally present in each patch (which varied slightly with the exact size of each patch). The six formulations are shown in the table below (w/w %):

| | 1 | 2 | 3 | 4 | 5 | 6 |
|---|---|---|---|---|---|---|
| Lumateperone | 20% | 20% | 20% | 20% | 20% | 20% |
| Lauryl Lactate | 14% | 14% | 14% | 14% | 15% | — |
| Propylene Glycol | 10% | 10% | 10% | 10% | 10% | — |
| DDAO | — | 1% | 2% | 3% | 2% | — |
| Lactic Acid | 1% | 1% | 1% | 1% | — | — |
| Ascorbyl Palmitate | 1% | 1% | 1% | 1% | 1% | — |
| Adhesive | Q.S. | Q.S. | Q.S. | Q.S. | Q.S. | Q.S. |
| Drug Delivery | 51% | 57% | 61% | 62% | 49% | 13% |

All six formulations show similar irritation and stability results: there is no significant irritation or discoloration of the skin after 72 hours of application (comparable to control) and the patches show <0.3% degradation on storage at room temperature for up to 2 weeks. On storage at room temperature for up to 2 months, the patches show degradation of 0.1 to 0.7%. Lumateperone is thus stable on room temperature storage in these patch formulations, and the patch formulations are not significantly irritating to the skin. Drug delivery results show that both lactic acid and DDAO enhance in vivo delivery with an unexpected synergistic effect compared to excipient-only patches. Patches lacking any excipient (adhesive only) show very poor drug delivery.

1. A transdermal pharmaceutical formulation comprising 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (Compound of Formula I) in free base form;
wherein the formulation is comprised in an adhesive patch; and wherein the formulation comprises an adhesive polymer;
wherein the formulation further comprises one or more excipients selected from lauryl lactate, isopropyl myristate, oleayl oleate, methyl laurate, isopropyl palmitate, and ethyl oleate.

2. (canceled)

3. (canceled)

4. (canceled)

5. The formulation of claim 1, wherein the formulation comprises from 1 to 1000 mg of the Compound of Formula I in free base form.

6. The formulation of claim 1, further comprising one or more excipients selected from the group consisting of solvents, solubilizers, plasticizers, surfactants, binders, humectants, antioxidants, buffering agents.

7. The formulation of claim 6, wherein the one or more excipients are selected from any of the following: alcohols, non-alcoholic solvents, gums, polysaccharides and polysaccharide derivatives, gelatins polyvinylpyrrolidones, polyethylene oxide and/or polypropylene oxide polymers and copolymers, polyacrylate polymers, polyamide polymers, sugars and sugar alcohols, polypeptides/proteins, amino acids, inorganic or organic acids and their salts and esters, inorganic or organic bases, anionic surfactants, cationic surfactants, zwitterionic surfactants, nonionic surfactants, sorbitan esters, polyethoxylated sorbitan esters, fatty acid esters, fatty alcohols, and antioxidants.

8. The formulation of claim 1, wherein the formulation comprises the adhesive polymer in an amount from 25-80% by weight.

9. The formulation of claim 8, wherein the adhesive polymer comprises a polymer selected from the group consisting of acrylate polymers or co-polymers; polyvinylpyrrolidones, copolymers of maleic acid or a maleic ester with a vinyl ether, cellulose derivatives, silicone polymers, and mixtures thereof.

10. The formulation of claim 1, wherein the formulation further comprises either dimethyldodecyl amine oxide or lactic acid.

11. The formulation of claim 1, wherein the formulation further comprises propylene glycol.

12. The formulation of claim 9, wherein the adhesive polymer comprises a mixture of a polyacrylate polymer and a silicone polymer.

13. A transdermal pharmaceutical device comprising the formulation according to claim 1, wherein the device comprises at least two layers, wherein at least one layer is a drug reservoir layer, and at least one layer is a backing layer, and wherein the drug reservoir layer comprises said formulation.

14. The device of claim 13, wherein the drug reservoir layer is the skin-contacting layer of the device.

15. The device of claim 13, wherein the device further comprises a third layer, separate from the drug reservoir layer, which third layer is the skin-contacting layer and which layer comprises an adhesive polymer, and which layer is substantially free of the Compound of Formula I prior to use of the device.

16. The device of claim 15, wherein the device further comprises an intermediate layer disposed between the skin contact layer and the drug reservoir layer, which layer is a rate-control layer.

17. The device of claim 13, wherein the skin-contacting layer comprise one or more excipients selected from lauryl lactate, isopropyl myristate, oleayl oleate, methyl laurate, isopropyl palmitate, and ethyl oleate.

18. The device of claim 13, wherein the device comprises from 1 to 1000 mg of the Compound of Formula I in free base form.

19. The device of claim 13, wherein the device is formulated to deliver the Compound of Formula I to a patient over a period of time from 1 to 30 days.

20. The device of claim 13, wherein the device is formulated to deliver the Compound of Formula I to a patient at an average daily dosage of from 1 to 60 mg per day (free base equivalent).

21. The device of claim 13, wherein the device systemically delivers a mean daily dose of the compound of Formula I of 0.1 to 5.0 mg per day (free base equivalent).

22. The device of claim 13, wherein the device delivers the compound of Formula I or the Compound of Formula II or the Compound of Formula III at a rate sufficient to maintain a steady state maximum plasma concentration of the compound (free base) of 5 to 50 ng/mL and/or sufficient to maintain a steady state 24-hour mean plasma concentration area under the curve (AUC) of the compound (free base) of 5 to 100 ng-hr/mL.

23. A method for the prophylaxis or treatment of a disease or abnormal condition involving or mediated by the 5-HT_{2A} receptor, serotonin transporter (SERT), and/or dopamine D₁/D₂ receptor signaling pathways, in a patient in need thereof, comprising administering to the patient by a transdermal route, a therapeutically effective amount of the Compound of Formula I by administering to said patient the formulation according to claim 1.

24. (canceled)

25. (canceled)

26. The method according to claim 23, wherein the disease or condition is selected from obesity, anorexia, bulimia, depression, anxiety, psychosis, schizophrenia, obsessive-compulsive disorder, sexual disorders, migraine, attention deficit disorder, attention deficit hyperactivity disorder, sleep disorders, conditions associated with cephalic pain, social phobias, dementia bipolar disorder, and bipolar depression.

27. A method for the prophylaxis or treatment of a disease or abnormal condition involving or mediated by the 5-HT_{2A} receptor, serotonin transporter (SERT), and/or dopamine D₁/D₂ receptor signaling pathways, in a patient in need thereof, comprising administering to the patient by a transdermal route, a therapeutically effective amount of Compound of Formula I by administering to said patient the device according to claim 12.

28. The method according to claim 27, wherein the disease or condition is selected from obesity, anorexia, bulimia, depression, anxiety, psychosis, schizophrenia, obsessive-compulsive disorder, sexual disorders, migraine, attention deficit disorder, attention deficit hyperactivity disorder, sleep disorders, conditions associated with cephalic pain, social phobias, dementia, bipolar disorder, and bipolar depression.

29. The formulation of claim 1, wherein the one or more excipients are selected from lauryl lactate and methyl laurate.

30. The formulation of claim 7, wherein the one or more excipients are selected from any of the following: ethanol, isopropanol, propanol, glycerol, propylene glycol, butylene glycol, pentylene glycol, hexylene glycol, polyethylene glycol, dimethylsulfoxide, dimethylformamide, acetonitrile, acacia gum, guar gum, agar, xanthan gum, tragacanth gum, karaya gum, gellan gum, starches, dextrans, pectins, alginates, carrageenans, cellulose, cellulose derivatives, bovine gelatins, porcine gelatins, avian gelatins, fish gelatins, poloxamers, carbopols, dextrose, lactose, galactose, glucose, ribose, sucrose, trehalose, mannitol, maltitol, lactitol, sorbitol, xylitol, erythritol, galactitol, inositol;
benzoic acid, toluenesulfonic acid, phosphoric acid, sulfuric acid, hydrochloric acid, tartaric acid, oxalic acid, cyclamic acid, ascorbic acid, methanesulfonic acid, benzenesulfonic acid, formic acid or their sodium, potassium, calcium, magnesium, lithium, ammonium salts; sodium lauryl sulfate, sodium laureth sulfate, sodium dodecylbenzenesulfonate, sodium lauroyl sarcosinate, sodium stearate, benzalkonium halides, cetylpyridinium halides, cetrimonium halides, benzethonium halides, cocamidopropyl betaine, polyethylene glycol polydodecyl ethers, sorbitan monolaurate, sorbitan monooleate, sorbitan monopalmitate, sorbitan monostearate, sorbitan tristearate, polysorbate 20, polysorbate 40, polysorbate 60, polysorbate 80, dodecanol, octyldecanol, lauryl alcohol, ascorbic acid, citric acid, ascorbyl palmitate, sodium metabisulfite, BHT, BHA, TBHQ, propyl gallate, beta-carotene, tocopherols, and tocotrienols.

31. The formulation of claim 28, wherein the cellulose derivatives are selected from carboxymethyl cellulose, methylcellulose, hydroxyethyl cellulose, hydroxypropyl cellulose, and hydroxypropyl methyl cellulose.

32. The formulation of claim 9, wherein the adhesive polymer comprises a polymer selected from the group consisting of acrylate polymers or co-polymers, linear polyvinylpyrrolidones, cross-linked polyvinylpyrrolidones, copolymers of maleic acid or anhydride with methyl vinyl ether, carboxymethyl cellulose, dimethylsilicone, and mixtures thereof.

33. The formulation of claim 1, wherein the formulation comprises about 10% w/w of the Compound of Formula I, and about 1% w/w of propylene glycol.

34. The formulation of claim 1, wherein the formulation comprises about 10% w/w of the Compound of Formula I, about 5% w/w of propylene glycol, about 5% by weight of lauryl lactate, about 5% w/w of methyl laurate, and about 5% w/w of propylene glycol dicaprate/dicaproate.

35. The formulation of claim 1, wherein the formulation comprises about 20% w/w of the Compound of Formula I, about 10% w/w of propylene glycol, about 15% w/w of lauryl lactate, and about 2% w/w of dimethyldodecylamine oxide.

36. The formulation of claim 1, wherein the formulation comprises about 20% w/w of the Compound of Formula I, about 10% w/w of propylene glycol, about 14% w/w of lauryl lactate, about 1% lactic acid, and about 0-3% w/w dimethyldodecylamine oxide.

37. The formulation of claim 36, wherein the formulation comprises 2% or 3% w/w dimethyldodecylamine oxide.
